(19) United States
(12) Patent Application Publication
(10) Pub. No.: US 2001/0037150 A1
(43) Pub. Date: Nov. 1, 2001

(54) Intraocular lens materials with low incidence of posterior capsule opacification

(76) Inventors: Kwan Y. Chan, Fort Worth, TX (US); Mutlu Karakelle, Fort Worth, TX (US); Reijo Linnola, Vaasa (FI)

Correspondence Address: Alcon Universal Ltd., c/o Alcon Research, Ltd., 6201 So. Freeway, Fort Worth, TX, 76134-2099, US

(21) Appl. No.: 09/827,245

(22) Filed: Apr. 5, 2001

Related U.S. Application Data
(60) Provisional application No. 60/260,553, filed on Jan. 9, 2001.
(60) Provisional application No. 60/195,765, filed on Apr. 10, 2000.

(51) Int. Cl. A61F 2/16

(52) U.S. Cl. 623/6.58; 623/6.62

(57) ABSTRACT

IOL bodies comprising materials having a Fibronectin/Vitronectin Compatibility Index>1 and a Lens Epithelial Cell Growth Biocompatibility Index≧1 on their posterior surface have a low incidence of posterior capsule opacification.

This application claims priority from co-pending U.S. Provisional Application Ser. No. 60/260,553 filed Jan. 9, 2001, and No. 60/195,765 filed Apr. 10, 2000.

### FIELD OF THE INVENTION

This invention relates to intraocular lenses. In particular, the present invention relates to biocompatible intraocular lens materials having a low incidence of posterior capsule opacification.

### BACKGROUND OF THE INVENTION

Foldable intraocular lens (“IOL”) materials can generally be divided into three categories: silicone materials, hydrogel materials, and non-hydrogel acrylic materials. Many materials in each category are known. See, for example, Foldable Intraocular Lenses, Ed. Martin et al., Slack Incorporated, Thorofare, N.J. (1993). Biocompatibility varies among different IOL materials within and among each category. Although the distinction between hydrogel and non-hydrogel acrylic materials is sometimes unclear, for purposes of the present application, acrylic materials that absorb 5% (by weight) or less water at 37° C. are considered non-hydrogel acrylic materials.

One measure of biocompatability for an IOL can be the incidence of posterior capsule opacification (“PCO”). A number or factors may be involved in causing and/or controlling PCO. For example, the design and edge sharpness of an IOL may be a factor. See, Nagamoto et al., J. Cataract Refract. Surg., 23:866-872 (1997); and Nagata et al., Jpn. J. Ophthalmol., 40:397-403 (1996). See, also, U.S. Pat. Nos. 5,549,670 and 5,693,094. Another factor appears to be the lens material itself. See, for example, Mandle, “Acrylic lenses cause less posterior capsule opacification than PMMA, silicone IOLs,” Ocular Surgery News, Vol. 14. No. 15, p. 23 (1996). See, also, Oshika, et al., “Two Year Clinical Study of a Soft Acrylic Intraocular Lens,” J. Cataract. Refract. Surg., 22:104-109 (1996); and Ursell et al., “Relationship Between Intraocular Lens Biomaterials and Posterior Capsule Opacification,” J. Cataract Refract. Surg., 24:352-360 (1998).

One method of addressing the PCO problem involves administering a pharmaceutical agent to the capsular bag area at the time of, or immediately after, extracapsular cataract extraction. See, for example, U.S. Pat. No. 5,576,345 (pharmaceutical agent=the cytotoxic agent taxol or an ophthalmically acceptable derivative); U.S. Pat. No. 4,515,794; and U.S. Pat. No. 5,370,687. Alternatively, the pharmaceutical agent may be tethered to the surface of the IOL material. See, for example, U.S. Patent No. 4,918,165. The pharmaceutical agents are intended to kill or prevent the growth of proliferating cells that might cause PCO or “secondary cataracts.” Yet another method involves the physical destruction or removal of lens epithelial cells. See, Saika et al., J. Cataract Refract. Surg., 23:1528-1531 (1997).

Another method of addressing PCO is the prophylactic laser therapy method disclosed in U.S. Pat. No. 5,733,276. According to this method, the lens capsule is irradiated with laser irradiation to destroy cells which remain in the lens capsule after extraction of a cataract.

Other methods theorized for reducing the risk of PCO involve adhering the posterior capsule to the IOL at the time of implantation, as in U.S. Pat. No. 5,002,571. According to the '571 patent, a non-biological glue or, preferably, a biological glue, such as fibrin, collagen, or mussel glue, is used to adhere the posterior lens capsule to the posterior surface of an IOL. The glue may be applied over the entire posterior surface of the IOL or just as an annulus around the outer perimeter of the posterior surface of the IOL.

In contrast, U.S. Pat. No. 5,375,611 discloses a method of reducing the risk of PCO by preventing the adherence of the posterior capsule to the IOL. According to the '611 patent, the posterior surface of the lens capsule itself is chemically modified at the time of extracapsular cataract extraction. The chemical modification is achieved by depositing a water-insoluble stable or permanent layer of a cell attachment preventing compound onto the posterior surface of the lens capsule. The stable or permanent layer may be a polymer, such as polyethylene glycol, polysaccharides, polyethylenepropylene glycol, and polyvinyl alcohols.

### SUMMARY OF THE INVENTION

The present invention relates to intraocular lens (“IOL”) materials having a low incidence of posterior capsule opacification (“PCO”). The materials of the present invention adsorb fibronectin and vitronectin at a combined level greater than ACRYSOF® MA30BA IOL, which is used as a standard. Additionally, the materials of the present invention have a Lens Epithelial Cell Growth Biocompatibility Index≧1. In one embodiment, the entire IOL optic consists of the materials of the present invention. Alternatively, the posterior surface of the IOL optic is formed or coated with the materials of the present invention, with the remainder of the optic comprising an ophthalmically acceptable lens material.

Without intending to be bound by any theory, it is believed that IOL posterior surfaces that specifically and strongly bind to the lens capsule, whether directly through vitronectin and fibronectin or through such adhesive proteins and a single or small lens epithelial cell layer, significantly reduce the risk of or prevent PCO.

### DETAILED DESCRIPTION OF THE INVENTION

According to the present invention, IOL materials having a low incidence of PCO are selected using both a fibronectin and vitronectin adhesive protein test and a lens epithelial cell growth test. Many IOL lens-forming monomers are known. See, for example, U.S. Pat. Nos. 5,290,892 and 5,331,073, the contents of both of which are hereby incorporated by reference. The IOL materials of the present invention preferably contain at least one aryl-containing hydrophobic acrylic material, meaning that the homopolymer of such monomer has an equilibrium water content of less than 3% as determined gravimetrically in deionized water at ambient conditions. Known IOL lens-forming monomers are combined using techniques known in the art to produce copolymeric materials meeting the elongation and Tg requirements below and then simply screened to determine if they satisfy the protein adsorption and lens epithelial cell growth tests below.

1. In Vitro Protein Adsorption Assay for Fibronectin and Vitronectin

Radiolabeled proteins (fibronectin or vitronectin, as the case may be) are used, preferably 125I-labeled. Unlabeled human fibronectin or vitronectin (each is evaluated separately) at 0.2 mg/ml BSS is mixed with appropriate volume of labeled protein to yield 0.2 μCi/ml radioactivity. IOL materials in the shape of IOL optics are incubated with above solution for 2 hr at 37° C. Afterwards, the optics are washed six times with BSS to remove unbound protein. The radioactivity of optics are counted in a gamma-counter, and converted to ng protein adsorbed per sq.cm of total surface area. For each test material, duplicate samples are prepared. One is evaluated using fibronectin and one using vitronectin, then the results are combined to give a total amount of adsorbed protein (fibronectin plus vitronectin) for that test material. A Fibronectin/Vitronectin Compatibility Index is determined by dividing the total ng protein (fibronectin plus vitronectin) adsorbed per sq.cm of total surface area for the tested optic by the total ng protein (fibronectin plus vitronectin) adsorbed per sq.cm of total surface area for an ACRYSOF® MA30BA IOL optic, which is used as a standard. The IOL materials of the present invention have a Fibronectin/Vitronectin Compatibility Index>1, preferably≧1.1.

2. In Vitro Cell Growth Assay for Rabbit Lens Epithelial Cells

An in vitro cell growth assay using rabbit lens epithelial cells is used to determine a Lens Epithelial Cell Growth Biocompatibility Index. A rabbit lens epithelial cell line (e.g. AG line) is used. Single cell suspensions are prepared in culture medium. The cells are mixed with 3H-thymidine (final radioactivity is 2 μCi/ml) in order to label DNA synthesis as an indicator of cell growth. In a 96-well plate, the IOL materials in the shape of IOL optics are placed inside the wells. The cells are plated to the wells at 10,000 cells per well, with labeled thymidine in culture medium. The cells are incubated for 1-2 days at 37° C. in a gas incubator with 95% air and 5% carbon dioxide. After the radioactive medium is removed, the cells are rinsed, treated with 10% trichloroacetic acid, solubilized with 1% sodium dodecyl sulfate, and processed for liquid scintillation counting in a beta-counter. The radioactivity is proportional to cell growth and is expressed as dpm (disintegration per min) per sq.cm of optic surface area being exposed to cells. Other methods of determining cell growth can be used, such as non-radioactive technique based on specific dye-staining of new DNA. Irrespective of the technique used to quantify cell growth, the cell growth assay consists of plating cells at low density on the IOL optics and allowing a few days for cell growth to proceed. At the end, cell growth is determined and expressed per surface area of the optics. The Lens Epithelial Cell Growth Biocompatibility Index is determined with reference to an ACRYSOF® MA30BA IOL optic as the standard. The individual method used to quantify cell growth is not critical, as long as the same method is used for both the test material and the ACRYSOF® MA30BA IOL optic standard. The Lens Epithelial Cell Growth Biocompatibility Index is the cell growth for the test IOL material divided by the cell growth for an ACRYSOF® MA30BA IOL optic standard. The IOL materials of the present invention have a Lens Epithelial Cell Growth Biocompatibility Index≧1, preferably≧1.1.

Also preferred are IOL materials which are substantially free of glistenings in a physiologic environment. Glistenings are the result of condensation of water vapor within the lens. Although glistenings have no detrimental effect on the function or performance of IOLs made from acrylic materials, it is nevertheless cosmetically desirable to minimize or eliminate them. IOL materials are substantially free of glistenings in a physiologic environment if they have an average of no more than approximately 1-2 glistenings per mm² when evaluated in the test described below. Preferably, the average number of glistenings per mm² will be much less than 1.

The presence of glistenings is measured by placement of a lens sample into a vial and adding deionized water or a balanced salt solution. The vial is then placed into a water bath preheated to 45° C. Samples are to be maintained in the bath for 24 hours. The sample is then placed either in a 37° C. bath or at room temperature and allowed to equilibrate for 2 hours. The sample is removed from the vial and placed on a microscope slide. Visualization of glistenings is done with light microscopy using a magnification of 50 to 200×.

The IOL materials of the present invention are also selected so that they possess the following T_{g}, and elongation properties, which make the materials particularly suitable for use in IOLs which are to be inserted through incisions of 5 mm or less.

The glass-transition temperature (“Tg”) of the IOL material, which affects the material's folding and unfolding characteristics, is preferably between about −20 to +25° C., and more preferably between about −5 and +1620 C. Tg is measured by differential scanning calorimetry at 10° C./min., and is determined at the midpoint of the transition of the heat flux curve.

The IOL material should also have an elongation of at least about 150%, preferably at least 200%, and most preferably about 300-600%. This property indicates that an IOL optic made of the material generally will not crack, tear or split when folded. Elongation of polymer samples is determined on dumbbell shaped tension test specimens with a 20 mm total length, length in the grip area of 4.88 mm, overall width of 2.49 mm, 0.833 mm width of the narrow section, a fillet radius of 8.83 mm, and a thickness of 0.9 mm. Testing is performed on samples at ambient conditions using an Instron Material Tester (Model No. 4442 or equivalent) with a 50 Netwon load cell. The grip distance is set at 14 mm and a crosshead speed is set at 500 mm/minute and the sample is pulled until failure. The elongation (strain) is reported as a fraction of the displacement at failure to the original grip distance.

In one embodiment, the entire IOL optic consists of the materials of the present invention. Alternatively, the anterior surface, posterior surface, or both of the IOL optic is coated with the materials of the present invention, with the remainder of the optic comprising an ophthalmically acceptable lens material. In the case where the materials of the present invention form a coating, with the remainder of the optic comprising any opthalmically acceptable intraocular lens material, the coating should be applied in a manner to form a coating of uniform thickness. The coating generally will be about 25 μm or less in thickness, preferably about 5 μm or less in thickness. The coating may be applied using known techniques, including solution and vapor deposition techniques.

The IOL material of the present invention preferably has a refractive index of at least about 1.50 as measured by an Abbe' refractometer at 589 nm (Na light source), particularly when the entire IOL optic consists of the materials of the present invention. IOL optics made from materials having a refractive index lower than 1.50 are necessarily thicker than optics of the same power which are made from materials having a higher refractive index. As such, IOL optics made from materials having a refractive index lower than about 1.50 generally require relatively larger incisions for IOL implantation.

The IOL bodies formed of the materials of the present invention or formed of other materials and coated in whole or in part with the materials of the present invention are preferably designed so that at least one of the optic's anterior and posterior surfaces forms a corner where it meets the optic's edge surface such that, at 150× magnification (of a cross-sectional view), the corner (i) is a sharp corner having an angle from 70-140°, more preferably 80-130°, and most preferably 90-120°, or (ii) is a round corner that has an arc that subtends an angle of 90° or less to the center of a circle having a radius ≦0.025 mm. As used herein, “optic” and “body” are used interchangeably and both mean the central part of the IOL incorporating the image-forming component of the IOL (see the definition of “body” in ISO/FDIS 11979-1:1999 (E)).

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

We claim:

1. An intraocular lens body having an anterior and a posterior surface separated by an edge, wherein the posterior surface comprises a material having a T_{g} of about −20 to +25° C., an elongation of at least about 150%, a Fibronectin/Vitronectin Compatibility Index>1, and a Lens Epithelial Cell Growth Biocompatibility Index≧1.

2. The intraocular lens body of claim 1 wherein the posterior surface comprises a non-hydrogel acrylic material and at least one of the anterior and posterior surfaces forms a corner where it meets the edge surface such that, at 150× magnification, the corner (i) is a sharp corner having an angle from 70-140° or (ii) is a round corner that has an arc that subtends an angle of 90° or less to the center of a circle having a radius ≦0.025 mm.

3. The intraocular lens body of claim 2 wherein the corner is a sharp corner having an angle from 80-130°.

4. The intraocular lens body of claim 3 wherein the corner is a sharp corner having an angle from 90-120°.

5. The intraocular lens body of claim 1 wherein the material is substantially free of glistenings.

6. The intraocular lens body of claim 1 wherein the material has a Lens Epithelial Cell Growth Biocompatibility Index≧1.1.

7. The intraocular lens body of claim 1 wherein the material has a Fibronectin/Vitronectin Compatibility Index≧1.1.

8. The intraocular lens body of claim 1 wherein the material has a refractive index of about 1.50 or greater.

9. The intraocular lens body of claim 1 wherein the material forms a coating of about 25 μm or less in thickness on the posterior surface.

10. The intraocular lens body of claim 1 wherein the posterior surface consists of a coating material of about 5 μm or less in thickness, wherein the coating material has a T_{g} of about −20 to +25° C., an elongation of at least about 150%, a Fibronectin/Vitronectin Compatibility Index>1, and a Lens Epithelial Cell Growth Biocompatibility Index≧1.

11. The intraocular lens body of claim 1 wherein the entire lens body consists of a material having a T_{g} of about −20 to +25° C., an elongation of at least about 150%, a Fibronectin/Vitronectin Compatibility Index>1, and a Lens Epithelial Cell Growth Biocompatibility Index≧1.
